(19) United States
(12) Patent Application Publication
(10) Pub. No.: US 2004/0267275 A1
(43) Pub. Date: Dec. 30, 2004

(54) Spinal implant holder and rod reduction systems and methods

(76) Inventors: John R. Cournoyer, Norfolk, MA (US); Carl G. Souza, Dighton, MA (US); Anthony R. Carlone, Bristol, RI (US); Michael S. Varieur, Portsmouth, RI (US); Conor McCrea, Dedham, MA (US); Stephen Johnson, Greenville, RI (US)

Correspondence Address: NUTTER MCCLENNEN & FISH LLP, WORLD TRADE CENTER WEST, 155 SEAPORT BOULEVARD, BOSTON, MA, 02210-2604, US

(21) Appl. No.: 10/607,438

(22) Filed: Jun. 26, 2003

(51) Int. Cl. A61F 2/02; A61F 2/44; A61F 5/04; A61B 17/56; A61F 2/30; A61F 2/00; A61B 17/60; A61F 9/00; A61F 11/00

(52) U.S. Cl. 606/99; 606/205; 623/11.11; 623/17.11; 623/908; 606/61; 606/53

(57) ABSTRACT

An implant holder is provided having first and second gripping arms, each having a proximal portion with a mating element formed thereon for selectively mating to a medical device, and a distal portion adapted to at least partially circumferentially engage and axially align with a rod-receiving member, e.g., a head, of a spinal implant. The spinal implant holder further includes an actuator mechanism coupled to the first and second gripping arms and effective to move the gripping arms between an open position, in which the gripping arms can be positioned around the rod-receiving head of a spinal implant, and a closed position, in which the gripping arms engage the rod-receiving head of the spinal implant. A rod reduction device is also provided and includes an elongate inserter tool having a proximal end and a distal end with a fastener-retaining member formed thereon and adapted to retain a fastener therein. A reduction cowl is disposed around the fastener-retaining member and is preferably biased to a proximal position with respect to the fastener-retaining member. The device further includes an external housing disposed around at least a portion of the elongated inserter tool and having a first member that is adapted to couple to the proximal end of each gripping arm on the spinal implant holder, and a second member that is movable with respect to the outer sleeve and that is effective to apply a distally-directed force to the reduction cowl to advance a spinal rod disposed between the reduction cowl and the rod-receiving head of a spinal implant engaged by the spinal implant holder.

### FIELD OF THE INVENTION

The present invention relates to methods and devices for use in spinal surgery, and in particular to spinal implant holders, rod reduction devices, and methods for using the same.

### BACKGROUND OF THE INVENTION

Spinal fixation devices are used in orthopedic surgery to align and/or fix a desired relationship between adjacent vertebral bodies. Such devices typically include a spinal fixation element, such as a relatively rigid fixation rod, that is coupled to adjacent vertebrae by attaching the element to various anchoring devices, such as hooks, bolts, wires, or screws. The fixation rods can have a predetermined contour that has been designed according to the properties of the target implantation site, and once installed, the instrument holds the vertebrae in a desired spatial relationship, either until desired healing or spinal fusion has taken place, or for some longer period of time.

Spinal fixation devices can be anchored to specific portions of the vertebra. Since each vertebra varies in shape and size, a variety of anchoring devices have been developed to facilitate engagement of a particular portion of the bone. Pedicle screw assemblies, for example, have a shape and size that is configured to engage pedicle bone. Such screws typically include a threaded shank that is adapted to be threaded into a vertebra, and a head portion having a rod-receiving element, usually in the form of a U-shaped slot formed in the head. A set-screw, plug, or similar type of fastening mechanism, is used to lock the fixation rod into the rod-receiving head of the pedicle screw. In use, the shank portion of each screw is threaded into a vertebra, and once properly positioned, a fixation rod is seated through the rod-receiving member of each screw and the rod is locked in place by tightening a cap or other fastener mechanism to securely interconnect each screw and the fixation rod.

While current spinal fixation systems have proven effective, difficulties have been encountered in mounting rods into the rod-receiving member of various fixation devices. In particular, it can be difficult to align and seat the rod into the rod receiving portion of adjacent fixation devices due to the positioning and rigidity of the vertebra into which the fixation device is mounted. Thus, the use of a spinal rod reduction device, also sometimes referred to as a spinal rod approximator, is often required in order to grasp the head of the fixation device and reduce the rod into the rod-receiving head of the fixation device.

While several rod reduction devices are known in the art, some tend to be difficult and very time-consuming to use. Accordingly, there is a need for improved rod reduction devices, implants for use with rod reduction devices, and methods for seating a spinal rod in a rod-receiving member of one or more spinal implants.

### SUMMARY OF THE INVENTION

The present invention generally provides a spinal implant holder including first and second gripping arms having a proximal portion with a mating element formed thereon for selectively mating to a medical device, and a distal portion adapted to at least partially circumferentially engage and axially align with a rod-receiving member of a spinal implant. In an exemplary embodiment, the mating element on the spinal implant holder comprises a luer lock formed on at least one of an inner surface or an outer surface of the proximal portion of the first and second gripping arms. The spinal implant holder can further include an actuator mechanism coupled to the first and second gripping arms and effective to move the gripping arms between an open position, in which the gripping arms can be positioned around the rod-receiving member of a spinal implant, and a closed position, in which the gripping arms engage the rod-receiving member of the spinal implant. A locking mechanism can be coupled to the actuator to lock the first and second gripping arms in the closed position. In use, the first and second gripping arms are preferably effective to mate to a rod reduction device used in spinal surgery, and to align the rod reduction device with the rod-receiving member of a spinal implant being engaged by the first and second gripping arms.

The first and second gripping arms can have a variety of configurations. In one embodiment, the first and second gripping arms are opposed to one another and each arm is elongated between a proximal end and a distal end. Each gripping arm can include an inner, substantially hemispherical surface, and in an exemplary embodiment, each gripping arm includes a grasping element formed on an inner surface thereof and effective to grasp a spinal implant. The grasping element can be, for example, a pin member adapted to be disposed within a corresponding detent formed in the rod-receiving member of a spinal implant. Alternatively, or in addition, each grasping element can be a channel adapted to engage a corresponding lip formed around the rod-receiving member of a spinal implant.

In another embodiment, the actuator includes a first handle member mated to the first gripping arm, and a second handle member mated to the second gripping arm. The first and second handle members can be pivotally coupled to one another and movable between a first, open position in which the gripping arms are in the open position, and a second, closed position in which the gripping arms are in the closed position. A locking mechanism can optionally be coupled to the first and second handle members to lock the first and second handle members in the closed position, thereby locking the gripping arms in the closed position. In an exemplary embodiment, the locking mechanism is a ratchet-type mechanism extending between the first and second handle members.

The present invention also provides a rod reduction device having an elongate inserter tool including a proximal end and a distal end having a fastener-retaining member formed thereon and adapted to retain a fastener therein. A reduction cowl can be disposed around and biased to a proximal position with respect to the fastener-retaining member. The rod reduction device can also include an external housing, disposed around at least a portion of the elongate inserter tool, which includes a first member adapted to couple to a rod-receiving member of a spinal implant, and a second member movable with respect to the first member and effective to apply a distally-directed force to the reduction cowl to advance a spinal rod disposed between the reduction cowl and the rod-receiving member of a spinal implant into the rod-receiving member of the implant. In use, the reduction cowl is effective to enable the fastener-retaining member to move a predetermined distance in a proximal-distal direction with respect to the reduction cowl during reduction of a spinal rod.

In one embodiment, the first member of the external housing comprises an outer sleeve having a substantially cylindrical shape and including a proximal end, a distal end, and an inner lumen extending therethrough to receive the elongate inserter tool, and the second member of the external housing comprises an elongate pusher shaft extending through the first member and having a distal end positioned adjacent to the reduction cowl. Preferably, the first and second members are threadably mated to one another. In another embodiment, the first member of the external housing includes a mating element effective to mate to a spinal implant holder that is effective to couple to a rod-receiving portion of a spinal implant. The mating element can be, for example, a luer lock formed on at least one of an inner surface or an outer surface of the first member of the external housing.

In other aspects, the present invention provides an inserter tool, is disposable through a rod reduction device, that includes a coupling member adapted to mate to a rod-receiving member of a spinal implant, and a pusher member axially adjustable with respect to the coupling member. The inserter tool includes a shaft having a fastener-retaining member formed on a distal end thereof and, optionally, a reduction cowl disposed around and preferably biased in a proximal direction with respect to the fastener-retaining member. The reduction cowl is effective to move distally in response to a distally-directed force from the pusher member, greater than a biasing force, to advance a spinal rod toward a spinal implant mated to the coupling member, and to allow the fastener-retaining member to move in a proximal-distal direction during reduction of a spinal rod. A spring can be disposed between the reduction cowl and the fastener-retaining member to bias the reduction cowl in a proximal direction.

In yet another embodiment of the present invention, a modular system is provided including an implant holder and rod reduction device. The implant holder includes first and second gripping members, each gripping member including a proximal portion having a mating element formed thereon, and a distal portion having a grasping element adapted to engage a rod-receiving member of a spinal implant. An actuating member is provided and is effective to move the first and second gripping members between an open position, in which the members can be positioned around at least a portion of the rod-receiving member of a spinal implant, and a closed position, in which the members engage the rod-receiving member of the spinal implant. The rod reduction device of the system includes a first member having a mating element formed thereon and adapted to removably mate to the mating element formed on each of the first and second gripping members, and a second member rotatably coupled to and axially movable with respect to the first member such that the second member is effective to reduce a rod extending between the first and second gripping members and the spinal implant being engaged by the gripping members into the rod-receiving member of the spinal implant.

Methods of using the devices of the present invention are also provided.

### BRIEF DESCRIPTION OF THE DRAWINGS

The invention will be more fully understood from the following detailed description taken in conjunction with the accompanying drawings, in which:

FIG. 1A is a perspective view of one embodiment of a spinal implant holder according to the present invention;

FIG. 1B is a side view of the spinal implant holder shown in FIG. 1A;

FIG. 2A is a top view of the gripping arms of the spinal implant holder shown in FIGS. 1A and 1B;

FIG. 2B is a perspective view of the gripping arms of the spinal implant holder shown in FIGS. 1A and 1B;

FIG. 3A is a perspective view of one embodiment of a rod reduction device in accordance with the present invention;

FIG. 3B is perspective view of the inserter tool of the rod reduction device shown in FIG. 3A;

FIG. 3C is a perspective view of the outer sleeve of the rod reduction device shown in FIG. 3A;

FIG. 3D is a perspective view of the pusher member of the rod reduction device shown in FIG. 3A;

FIG. 3E is a cross-sectional view of the rod reduction device shown in FIG. 3A;

FIG. 3F is a perspective view of another embodiment of a rod reduction device in accordance with the present invention;

FIG. 4 is a perspective view of the rod reduction device of FIG. 3A attached to the spinal implant holder of FIG. 1A in accordance with another embodiment of the present invention;

FIG. 5 is a perspective view of one embodiment of a spinal implant for use with the devices of the present invention; and

FIG. 6 is a perspective view of the devices shown in FIG. 4 in use attached to a spinal implant having a spinal rod extending therethrough.

### DETAILED DESCRIPTION OF THE INVENTION

The present invention provides a spinal implant holder that is adapted to engage a rod-receiving head of a spinal implant, and a rod reduction device that can optionally couple to the spinal implant holder and that is effective to reduce a rod disposed within or adjacent to the rod-receiving head of the spinal implant. The spinal implant holder and the rod reduction device are modular in that they can be used alone, or they can couple to one another. Moreover, each device can include modular features that allow the devices to be used with a variety of spinal implants and other tools.

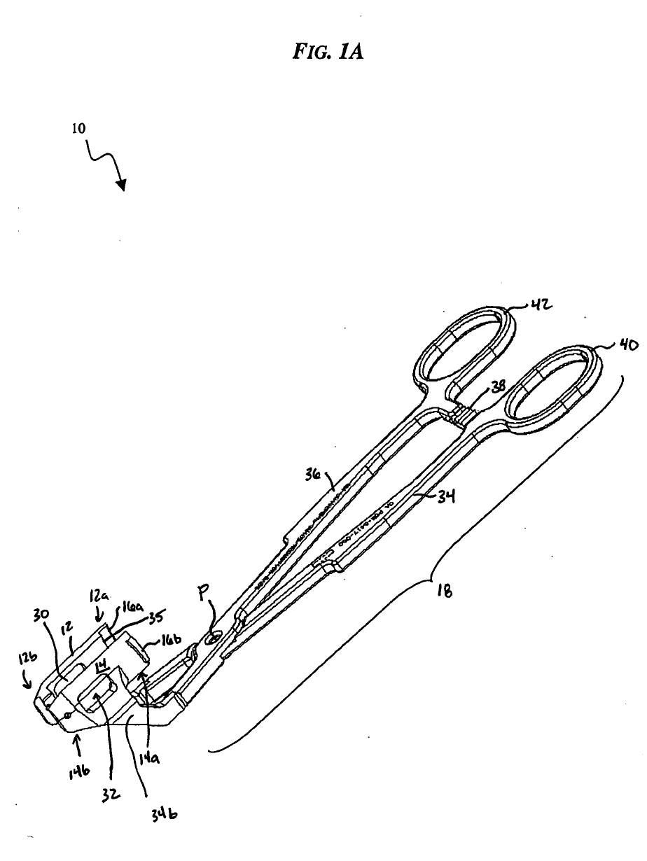

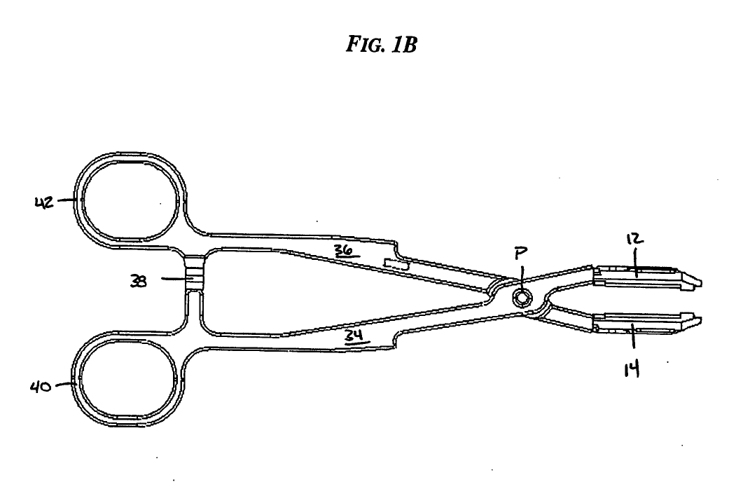

FIGS. 1A-1B illustrate one embodiment of a spinal implant holder 10 in accordance with present invention. As shown, the implant holder 10 generally includes first and second gripping arms 12, 14, each having a proximal portion 12a, 14a with a mating element 16a, 16b formed thereon for selectively mating to a medical device, and a distal portion 12b, 14b adapted to at least partially circumferentially engage and axially align with a rod-receiving member, e.g., a head, of a spinal implant. The spinal implant holder 10 further includes an actuator mechanism 18 coupled to the first and second gripping arms 12, 14 and effective to move the gripping arms 12, 14 between an open position, in which the gripping arms 12, 14 can be positioned around the rod-receiving head of a spinal implant, and a closed position, in which the gripping arms 12, 14 engage the rod-receiving head of the spinal implant.

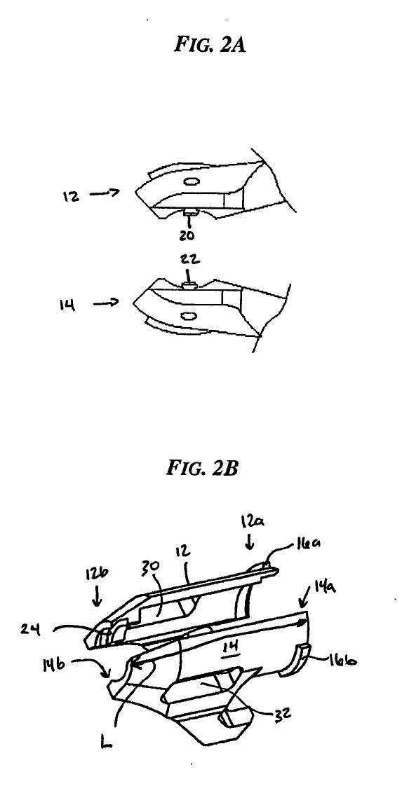

The first and second gripping arms 12, 14, which are shown in more detail in FIGS. 2A and 2B, can have a variety of configurations, but they should be adapted to securely grasp a rod-receiving head of the spinal implant. In an exemplary embodiment, as shown, each gripping arm 12, 14 has a generally elongated, hemispherical shape extending between a proximal end 12a, 14a and a distal end 12b, 14b. The arms 12, 14 are preferably positioned substantially parallel to, but spaced apart from, one another, as shown in FIG. 1B, to allow an implant to be engaged therebetween. The hemispherical surface that forms each arm 12, 14 can be substantially solid, or alternatively each arm 12, 14 can include a window 30, 32 formed therein to provide visual access to the head of an implant being engaged by the arms 12, 14. Each arm 12, 14 preferably has a length L that allows the distal end 12b, 14b of the arms 12, 14 to be positioned and secured around the head of a spinal implant, and the proximal end 12a, 14a of the arms 12, 14 to extend proximally from the proximal-most end of the head of the spinal implant to form a guide-like extension portion. This proximal, guide-like extension portion can serve as a guide for a variety of devices, or alternatively it can allow the proximal end 12a, 14a of the gripping arms 12, 14 to optionally couple to a rod reduction device, as will be discussed in more detail below.

In order to mate the distal end 12b, 14b of each gripping arm 12, 14 to the rod-receiving head of a spinal implant, each gripping arm 12, 14 preferably has a shape that is adapted to match the contour of the rod-receiving head of the spinal implant. As shown, for example, the distal portion 12b, 14b of the gripping arms 12, 14 can taper toward one another to fit around the rod-receiving head of an implant. Each gripping arm 12, 14 can also optionally include a grasping element formed on an inner surface thereof that is effective to grasp the spinal implant. Virtually any grasping element can be used, but in an exemplary embodiment, shown in FIG. 2A, a pin member 20, 22 is formed on an inner, distal surface of each gripping arm 12, 14 and it is adapted to be disposed within a corresponding detent formed in the rod-receiving head of a spinal implant. In another embodiment, shown in FIG. 2B, the grasping element is a channel (only one channel 24 is shown) that is adapted to engage a corresponding lip formed around the rod-receiving head of a spinal implant. This configuration is preferably used with polyaxial implants since it can be difficult to align a pin member with a corresponding detent formed in the rod-receiving head of a polyaxial implant. A person skilled in the art will appreciate that a variety of techniques can be used to mate the implant holder 10 to the rod-receiving head of a spinal implant.

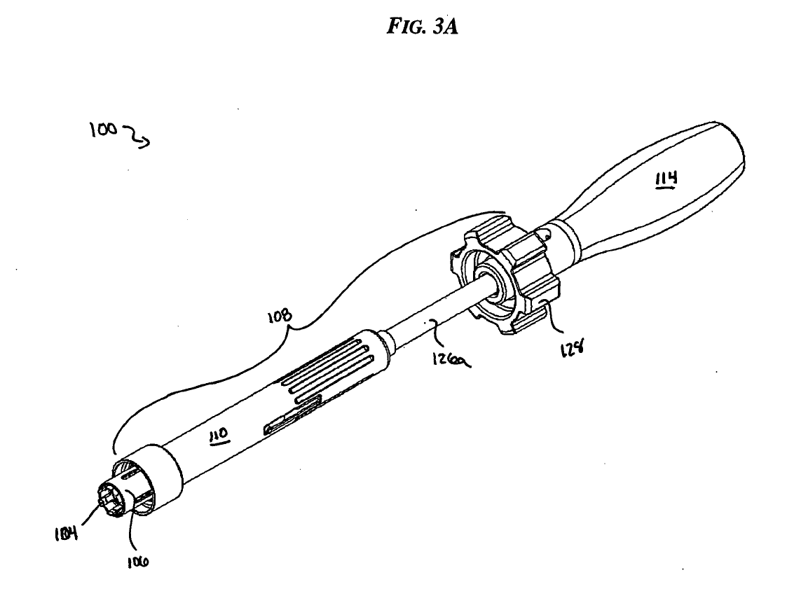

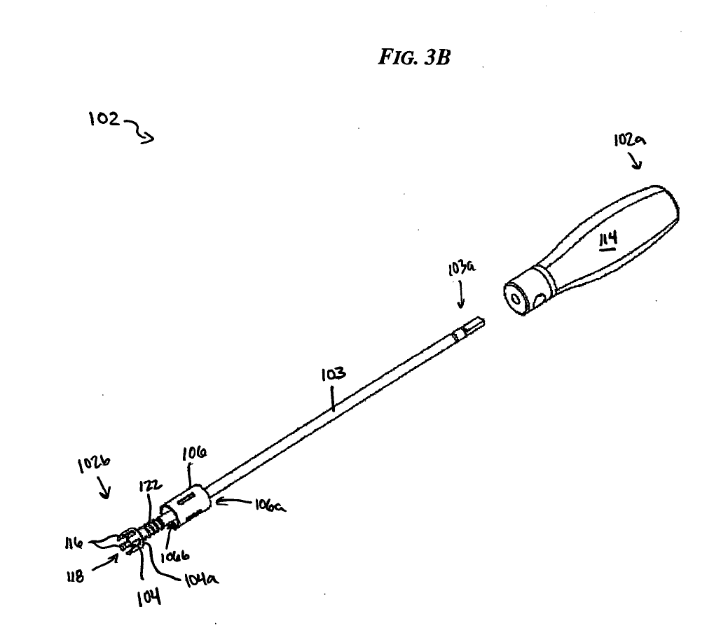

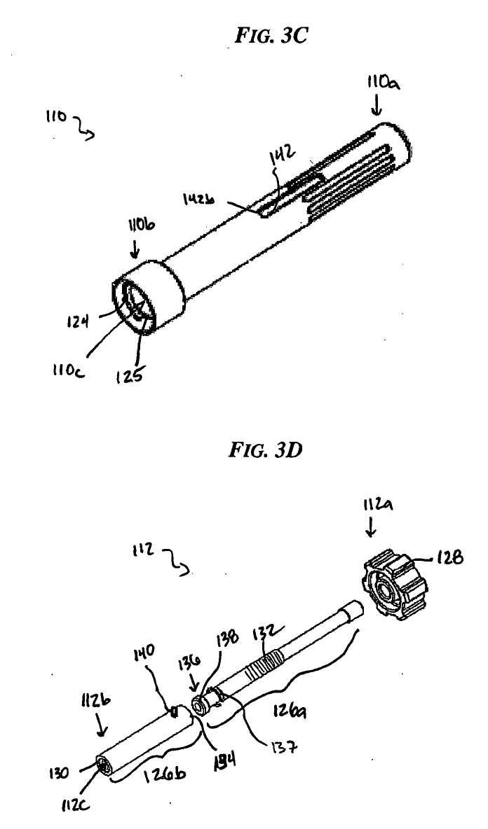

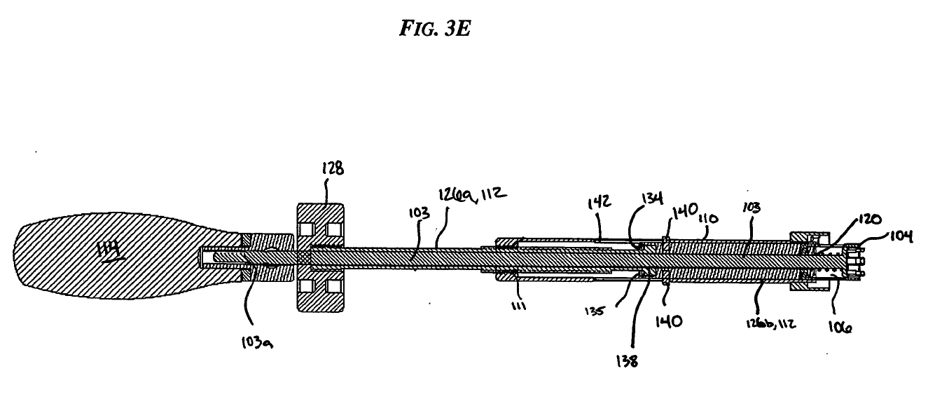

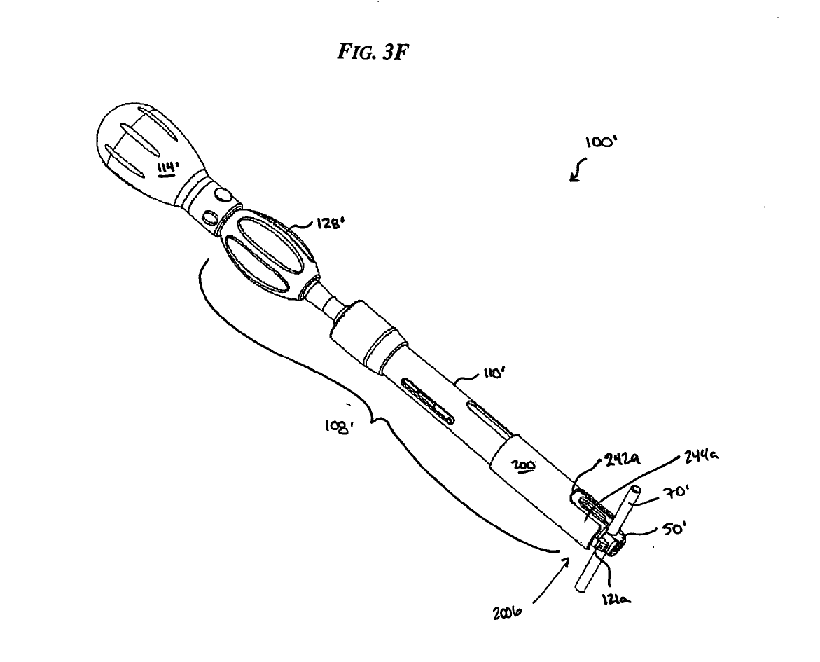

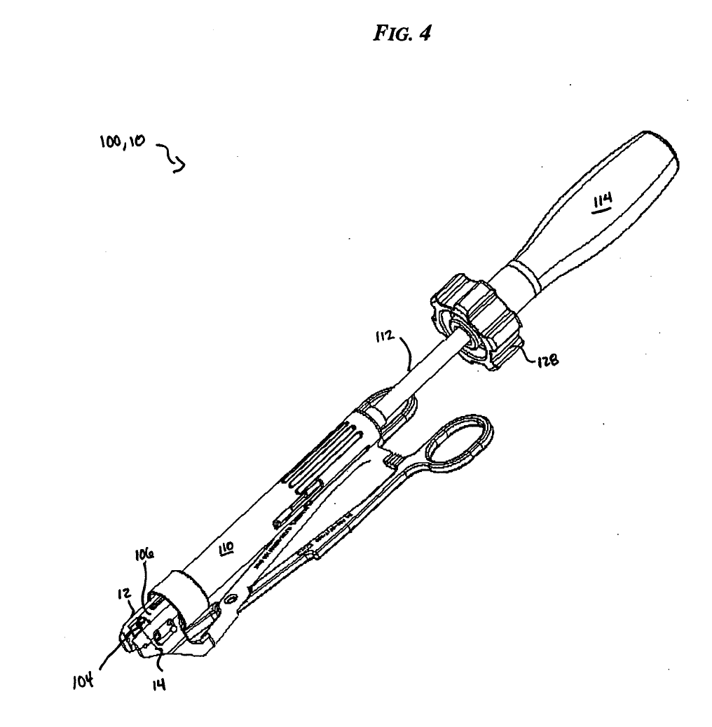

The proximal portion 12a, 14a of each gripping arm 12, 14 can also have a variety of configurations. In an exemplary embodiment, however, the proximal portion 12a, 14a of each gripping arm 12, 14 is adapted to mate to a rod reduction device, such as rod reduction device 100 described below with respect to FIGS. 3A-3E. While virtually any mating element can be used, in an exemplary embodiment the proximal end 12a, 14a of each gripping arm 12, 14 includes an annular lip 16a, 16b on an inner surface, or more preferably an outer surface, thereof. The annular lip 16a, 16b forms a male luer lock element which mates with a corresponding female luer lock element formed on a portion of the rod reduction device. Again, a person skilled in the art will appreciate that a variety of techniques can be used to mate the gripping arms 12, 14 of the spinal implant holder to a rod reduction device.

In use, the gripping arms 12, 14 are preferably movable between an open position (not shown), in which the gripping arms 12, 14 can be positioned around the rod-receiving head of a spinal implant, and a closed position (FIGS. 1A-2B), in which the gripping arms 12, 14 engage the rod-receiving head of the spinal implant. As indicated above, the gripping arms 12, 14 can be moved between the open and closed positions using an actuator mechanism 18 that is coupled to the first and second gripping arms 12, 14. While the actuator mechanism 18 can have a variety of configurations, FIGS. 1A-1B illustrate an exemplary embodiment of an actuator mechanism 18 having a first handle member 34 mated to or integrally formed with the first gripping arm 12, and a second handle member 36 mated to or integrally formed with the second gripping arm 14. The first and second handle members 34, 36 are pivotably coupled to one another at pivot point P and are movable between a first, open position in which the gripping arms 12, 14 are spaced apart from one another, and a second, closed position (as shown) in which the gripping arms 12, 14 are parallel to one another. Each handle member 34, 36 can include a finger loop 40, 42 formed on a proximal end thereof to facilitate grasping of the handle members 34, 36.

The handle members 34, 36 can be mated to the gripping arms 12, 14 at a variety of locations, but in an exemplary embodiment the handle members 34, 36 are preferably positioned to be offset with respect to the gripping arms 12, 14 to enhance surgeon visibility and access. As shown in FIG. 1A, the handle members 34, 36 can include a connecting portion (only one portion 34b is shown) that extends substantially transverse to the remaining portion of each handle member 34, 35 and that mates to the corresponding gripping arms 12, 14.

The actuator mechanism 18 can also optionally include a locking mechanism 38 coupled to the first and second handle members 34, 36 and effective to lock the first and second handle members 34, 36 and the gripping arms 12, 14 in the closed position. While virtually any locking mechanism 38 can be used, FIGS. 1A-1B illustrate a ratchet-type mechanism 38 extending between the first and second handle members 34, 36. The ratchet mechanism 38 locks the handle members 34, 36 in predetermined increments as the handle members 34, 36 are moved toward one another.

As indicated above, the spinal implant holder 10 can be used alone or alternatively it can be used in conjunction with a rod reduction device. By way of non-limiting example, the spinal implant holder 10 can serve as a guide for an inserter tool that is effective to apply one or more fasteners to a spinal implant. In an exemplary embodiment, however, the spinal implant holder 10 is used with the rod reduction device 100 shown in FIGS. 3A-3E.

As shown in FIGS. 3A-3E, the rod reduction device 100 generally includes two components: (1) an external housing 108 that is effective to couple to an implant and to reduce a rod disposed within the rod-receiving head of the implant, and (2) an inserter tool 102 that extends through the external housing 108 and that is effective to apply a fastener to an implant. The external housing 108 is formed from two members that work together with the inserter tool 102 to grasp the implant and to reduce the rod disposed within the head of the implant. The first member is an outer sleeve 110 (FIG. 3C) that has a distal end 110b that can couple to the head of an implant, and the second member is a pusher shaft 112 (FIG. 3D) that extends through and is threadably coupled to the outer sleeve 110. The inserter tool 102, which extends through the pusher shaft 112 (which in turn extends through the outer sleeve 110), includes a reduction cowl 106 disposed around its distal end. The reduction cowl 106 is configured to be positioned just distal to a distal end 112b of the pusher shaft 112. Thus, in use, as the pusher shaft 112 is threaded with respect to the outer sleeve 110, the pusher shaft 112 moves distally to apply a distally-directed force to the reduction cowl 106, thereby moving the reduction cowl 106, and consequently the inserter tool 102, distally. Eventually, the reduction cowl 106 comes into contact with a rod disposed within the head of an implant being engaged by the outer sleeve 110, thereby reducing the rod into the rod-receiving recess. Once the rod is fully reduced within the head of the implant, the inserter tool 102 can be used to apply a pre-loaded fastener to the head of the implant to lock the rod with respect to the implant.

The inserter tool 102, which is shown in more detail in FIG. 3B, can have a variety of configurations, but it preferably includes an elongate shank 103 extending between a proximal, handle member 114, and a distal, fastener-retaining member 104. The handle member 114, which is adapted to facilitate grasping of the tool 102, can have virtually any shape and size, and can be integrally formed with the elongate shank 103, or fixedly or removably attached to the elongate shank 103. The handle member 114 should, however, mate to the shank 103 such that rotation of the handle member 114 is effective to rotate the shank 103. The fastener-retaining member 104 that is formed on the distal end 102b of the inserter tool 102 is adapted to retain a fastener mechanism therein, and thus its configuration will vary depending on the configuration of the fastener to be retained therein. In an exemplary embodiment, shown in FIG. 3B, the fastener retaining member 104 is a cylindrical member having prongs 116 extending distally therefrom that define a socket 118. The socket 118 is effective to receive the fastener and the prongs 116 are effective to engage the fastener until such time that the fastener is mated to a spinal implant. In another embodiment, the fastener retaining member can include an engagement feature that is effective to mate to and engage and fastener. The engagement feature can optionally be in the form of an internal drive feature that is effective to drive the fastener, as well retain the fastener therein. In yet another embodiment, the inserter tool 102 is modular in that the fastener-retaining member 104 can be removably mated to the distal end 102b of the inserter tool 102 to allow several different fastener-retaining members to be attached to the tool 102.

The inserter tool 102 can also include a reduction cowl 106, which is slidably disposed around the fastener-retaining member 104 and, preferably, biased to a proximal position with respect to the fastener-retaining member 104. The reduction cowl 106 can have a variety of configurations, but in an exemplary embodiment it has a hollow, generally cylindrical shape with an open distal end 106b and a proximal end 106a having a bore 120 (FIG. 3E) formed therein to slidably receive the elongate shank 103 of the inserter tool 102. A spring 122 or similar biasing element is disposed within the reduction cowl 106 and abuts a proximal surface 104a of the fastener-retaining member 104. The spring 122 biases the reduction cowl 106 to a proximal position with respect to the fastener-retaining member 104. The reduction cowl 106 can also optionally include a stop member (not shown) formed thereon or disposed on the shank 103 of the inserter tool 102 to prevent proximal movement of the reduction cowl 106 beyond a predetermined position. The stop member can have virtually any configuration and can be, for example, a clamp member disposed around the shank 103 of the inserter tool 102.

The proximal biasing of the reduction cowl 106 allows the cowl 106 to remain in a proximal position while a fastener is loaded into the fastener-retaining member 104. During use of the device 100, the cowl 106 moves distally in response to a distally-directed force, generated by the pusher shaft 112, and that is greater than biasing force. In use, the reduction cowl 106 is preferably configured to travel distally to a position that is slightly distally beyond the fastener-retaining member 104 to allow the fastener-retaining member 104 to “float” with respect to the reduction cowl 106. In other words, the fastener-retaining member 104 can be moved in a proximal-distal direction, while the reduction cowl 106 maintains the rod in the reduced position. This allows the fastener disposed within the fastener-retaining member 104 to be aligned with the fastener-receiving member formed in the head of the spinal implant. As a result, the risk of cross threading between the fastener and the head of the implant is greatly reduced, if not eliminated. Moreover, this reduces or prevents the risk of damage to the implant during reduction. This configuration also allows either the reduction cowl 106 alone, or the reduction cowl 106 and the fastener-retaining member 104, to contact the spinal rod to reduce the rod within the rod-receiving head of a spinal implant. A person skilled in the art will appreciate that the device 100 can be provided within a reduction cowl 106, and that the fastener-retaining member 104, with a fastener (not shown) disposed therein, can be configured to reduce a rod.

As indicated above, the rod reduction device 100 further includes an outer housing 108 having a first member that is adapted to couple to a rod-receiving head of a spinal implant, and a second member that is disposed within the first member and that is movable with respect to the first member to apply a distally-directed force to the reduction cowl 106 to reduce a spinal rod disposed between the reduction cowl 106 and the rod-receiving head of a spinal implant. The first member can have a variety of configurations, but preferably it is in the form of an outer sleeve 110 (FIG. 3C) that has a generally elongate, cylindrical shape with a proximal end 110a, a distal end 110b, and an inner lumen 110c extending therethrough to receive the second member, e.g., pusher shaft 112. The proximal end 110a preferably includes internal threads 111 formed therein for mating with corresponding external threads 132 formed on the pusher shaft 112, as will be discussed in more detail below.

The distal end 110b of the sleeve 110 is preferably adapted to couple to the first and second gripping arms 12, 14 on the spinal implant holder 10. The distal end 110b of the sleeve 110 can thus include a mating element, such as a female luer lock element 124, formed therearound or therein for mating with the male luer lock element 116a, 116b formed on the gripping arms 12, 14. As shown in FIG. 3C, the luer lock element 124 is formed within the distal end 110b, thus the distal end 110b is enlarged to fit around the gripping arms 12, 14. In an exemplary embodiment, the luer lock element 124, 116a, 116b on the sleeve 110 and on the gripping arms 12, 14 is positioned such that a portion of the sleeve 110 extends around a substantial portion of the gripping arms 12, 14. This is effective to prevent misalignment between the rod reduction device 100 and the implant holder 10, and to prevent the arms 12, 14 from becoming disengaged from the implant. The distal end 110b of the outer sleeve 110 can also include an inner annular ring member 125 that is adapted to abut ridges 35 (FIG. 1A) formed around the proximal end 12a, 14a of each gripping arm 12, 14. The annular ring member 125 and ridges 35 are effective to prevent over-rotation of the outer sleeve 110 with respect to the gripping arms 12, 14. It also centers the rod reduction device 100 with the implant holder and prevents the gripping arms 12, 14 from splaying inward.

In an alternative embodiment, the distal end 110b of the sleeve 110 can be adapted to mate directly to the head of a spinal implant, rather than to the holder 10. By way of non-limiting example, FIG. 3F illustrates another embodiment of a rod reduction device 100′ having a sleeve 110′ that is adapted to engage the head of a spinal implant 50′. The sleeve 110′ (only a portion of which is shown) includes first and second branches (only one branch 121a is shown) that extend longitudinally from a distal end (not shown) thereof. Each branch 121a is substantially flexible and is biased to a closed, gripping position to allow the branches 121a to expand around the implant head 50′ to grip the implant. Each branch 121a also preferably includes a mating element, such as a protrusion, formed on an inner surface thereof to mate with a corresponding mating element, such as a detent or recess, formed in the head 50′ of the implant. Once the branches 121a are mated to the head 50′ of an implant, an outer sleeve 200 is preferably provided to prevent the branch members 121a from becoming disengaged with the implant head 50′. The outer sleeve 200 can have a variety of configurations, but it is preferably movable between proximal and distal positions. In the proximal position the outer sleeve 200 is raised above the flexible portion (not shown) of each branch member 121a to allow the branches 121a to expand to grip the implant. In the distal position the outer sleeve locks the branches 121a to the implant head 50′. The distal end 200b of the outer sleeve 200 also preferably includes opposed U-shaped channels (only one channel 242a is shown) that are configured to allow the device 100′ to capture a spinal rod 70′ above the head 50′ of the spinal implant while gripping the implant. The arms (only one arm 244a is shown) located between the U-shaped channels 242a are adapted to slide over the branches 121a when the sleeve 110′ is advanced to the distal position to lock the branches to the implant.

Still referring to FIG. 3F, in order to allow the sleeve 110′ to be disposed around and to mate to the rod-receiving head 50′ of an implant, the reduction cowl 106′ should be adapted to extend between the legs of the U-shaped rod-receiving head. In particular, rather than having a cylindrical shape, the reduction cowl 106′ preferably includes two finger-like members (not shown) that are adapted to extend between the legs of the U-shaped rod-receiving head 50′. The distal end of each finger-like member can optionally include a concave surface to facilitate reduction of a spinal rod by the reduction cowl 106′.

A person skilled in the art will appreciate that a variety of techniques can be used to mate the sleeve directly to a spinal implant, and that the sleeve and reduction cowl can each have a variety of configurations, shapes, and sizes.

Referring now to FIG. 3D, the second member of the outer housing 108 can also have a variety of configurations. In an exemplary embodiment, as shown, the second member is an elongate pusher shaft 112 having a proximal end 112a, a distal end 112b, and an inner lumen 112c extending therebetween to slidably receive the shank 103 of the inserter tool 102. The pusher shaft 112 can be formed from a single, integral component, but preferably the pusher shaft 112 includes separate proximal and distal portions 126a, 126b. The pusher shaft 112, as noted above and as shown in FIG. 3E, is adapted to be received within the inner lumen 110c of the sleeve 110.

The proximal portion 126a of the pusher shaft 112 preferably includes a knob 128 formed thereon or mated thereto. The knob 128 can have virtually any shape and size, but it should be adapted to assist in grasping and manipulating the device 100. The proximal portion 126a further includes threads 132 formed externally thereon to provide controlled axial movement of the pusher shaft 112 with respect to the outer sleeve 110. The threads 132, as shown in FIG. 3D, preferably extend along a substantial portion of the shaft 112, and are configured to mate with corresponding threads 111 formed within the proximal end 110a of the outer sleeve 110. As a result, controlled axial movement of the pusher shaft 112 can be achieved by rotating the pusher shaft 112, using knob 128, with respect to the outer sleeve 110.

The distal portion 126b of the pusher shaft 112 can be formed integrally with the proximal portion 126a, as indicated above. However, in one embodiment, it is a separate component that is axially secured to, but freely rotatable with respect to the proximal portion 126a. This allows the proximal portion 126a to rotate with respect to the sleeve 110, while the distal portion 126b moves distally to apply a force to the reduction cowl 106 without rotating against the reduction cowl 106. A variety of techniques can be used for connecting the proximal and distal portions 126a, 126b to allow rotatable movement therebetween. By way of non-limiting example, the distal portion 126b can include a proximal-surface 134 with a bore 135 (FIG. 3E) formed therein for receiving the proximal portion 126a, and the distal end 136 of the proximal portion 126a can include an annular lip or rim 138 that is adapted to abut the proximal surface 134. Thus, the proximal portion 126a can be inserted through the distal end 112b of the distal portion 126b and the annular lip or rim 138 will prevent the proximal portion 126a from sliding completely through the distal portion 126b. A clamp 137 or similar member can be provided to prevent slidable movement between the proximal and distal portions 126a, 126b of the pusher shaft 112.

In use, the distal portion 126b is preferably configured to apply a force to the reduction cowl 106 to reduce a rod disposed within a rod-receiving head of a spinal implant. Accordingly, the distal portion 126b of the pusher shaft 112 is preferably enlarged to provide a distal-most surface 130 that is adapted to abut the proximal end 106a of the reduction cowl 106. In an exemplary embodiment, the distal-most surface 130 has a shape and size corresponding to the shape and size of the proximal end 106a of the reduction cowl 106 to distribute the forces evenly around the reduction cowl 106.

The distal portion 126b of the pusher shaft 112 can also include an anti-rotation mechanism to prevent rotational movement of the distal portion 126b during use of the device 100. While a variety of techniques can be used to prevent rotational movement, an example of such a mechanism is at least one pin member 140 that is configured to extend into at least one longitudinal slot 142 formed in the outer sleeve 110. The longitudinal slot 142 will allow the pusher shaft 112 to move in a proximal-distal direction, yet the pin member will prevent rotational movement of the distal portion 126b of the pusher shaft 112 with respect to the outer sleeve 110. The pin member 140 and the slot 142 can also serve as a guide to indicate when reduction is complete. In particular, the slot 142 can have a length such that the pin member 140 will abut the distal end 142b of the slot 142 when reduction is complete.

FIG. 3E illustrates a cross-sectional view of the rod reduction device 100 in the fully assembled state. The device 100 can be assembled by inserting the proximal portion 126a of the pusher shaft 112 through the distal end 112b of the distal portion 126b of the pusher shaft 112. The clamp 137 is then preferably attached to the shaft 112 to prevent movement between the proximal and distal portions 126a, 126b. The proximal end 103a of the inserter tool 102 can then be introduced through the lumen 112c formed in the distal end of the pusher shaft 112. The outer sleeve 110 is then passed over the proximal ends 103a, 112a of the inserter tool 102 and the pusher shaft 112, and the bore 113 in the proximal end surface 111 of the outer sleeve 110 is then threaded onto the proximal portion 126a of the pusher shaft 112. Handle member 128 can then be fixedly attached to the proximal end 112a of the proximal portion 126a of the pusher shaft 112, and handle member 114 can then be attached to the proximal end 103a of the inserter tool 102. The pin member(s) 140 on the distal portion 126b can be welded to the distal portion 126b of the pusher shaft 112 after the device is fully assembly, or they can optionally be depressible to allow the sleeve 110 to be slid over the distal portion 126b.

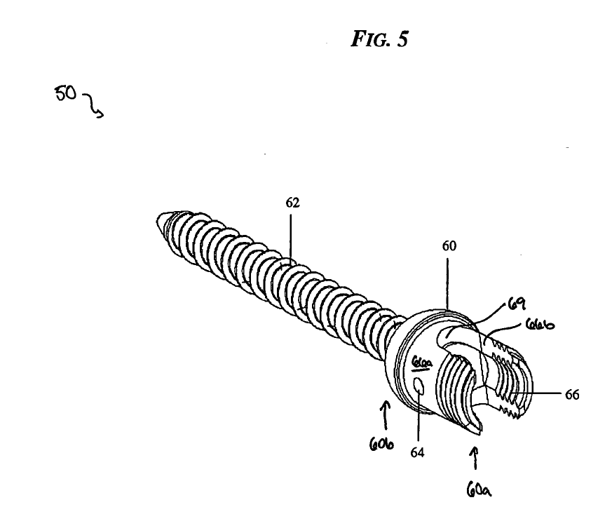

In use, the rod reduction device 100 can be coupled to the spinal implant holder 10, as shown in FIG. 4. The spinal implant holder 10 is first secured to the implant, and then the female luer lock element 124 formed within the outer sleeve 110 is mated to the male luer lock element 116a, 116b formed around the first and second gripping arms 12, 14 of the implant holder 10. When the gripping arms 12, 14 are mated to a spinal implant, the pusher shaft 112 can be rotated using knob 128 to apply a distal force to the reduction cowl 106, which is positioned between the gripping arms 12, 14. Since the outer sleeve 110 is engaged to the gripping arms 12, 14, which are mated securely to the implant, the reduction cowl 106, and consequently the inserter tool 102, can be moved distally to reduce a rod disposed within the rod-receiving head of the spinal implant. The fastener secured inside the inserter fingers 118 will contact the rod first. If the force to reduce the rod is too high, the reduction cowl 106 will contact the rod, and both the fastener and the reduction cowl 106 will reduce the rod. Once the rod is fully reduced, handle 114 of the inserter tool 102 can be rotated to apply the fastener (not shown) disposed within the fastener-receiving member 104 to the implant to fix the rod within the implant.

The implant holder 10 and the rod reduction device 100 can be used with a variety of spinal implants. To facilitate use with implants having different configurations, the rod reduction device 100 and/or the implant holder 10 can be in the form of a modular kit, which contains several modular inserter tools configured to mate with each of the different spinal implants, and multiple or modular implant holders having mating elements configured to mate with each of the different spinal implants, and different modular handles to satisfy the surgeon's needs.

FIG. 5 illustrates an exemplary embodiment of a spinal implant 50 for use with the rod reduction device 100 and spinal implant holder 10 according to the present invention. As shown, the implant 50 includes a threaded shank 62 and a generally U-shaped head 60 having an open proximal end 60a, and distal end 60b attached to the shank 62. Preferably, the shank 62 is rotatably mated to the distal end 60b of the head 60 to allow rotation of the head 60, e.g., the screw 50 is a polyaxial screw. A variety of techniques can be used to allow rotation of the head 60 with respect to the shank 62. By way of non-limiting example, the shank 62 can include an enlarged proximal portion (not shown) that can be disposed through a bore formed in the distal end 60b of the head 60. The enlarged proximal portion will prevent the shank 62 from extending completely through the bore. A compression cap (not shown) can be disposed within the head 60 to prevent the U-shaped head 60 from moving longitudinally along the threaded shank 62. Once a spinal rod is disposed within the U-shaped head 60 and secured by a closure mechanism, the rod and the compression cap will prevent rotation of the head 60 with respect to the shank 62.

The U-shaped head 60 includes opposed side walls 66a, 66b that define a rod-receiving portion 69 for seating a spinal fixation rod, and that are substantially parallel to one another. The outer surface of the head 60 includes opposed recesses (only one recess 66 is shown) formed therein for mating with the corresponding protrusions 20, 22 (FIG. 2A) formed on the inner surface of each gripping arm 12, 14. The inner surface of the head 60 includes threads 66 formed thereon for mating with a corresponding threads formed on a closure mechanism. While a threaded closure mechanism is illustrated, a person skilled in the art will appreciate that a twist-lock mechanism or other techniques can be used to mate a fastener to the head of the implant. Moreover, a person skilled in the art will appreciate that the implant 50 can have a variety of other configurations, and that the features illustrates can be used on a variety of implants, including hooks, bolts, wires, etc.

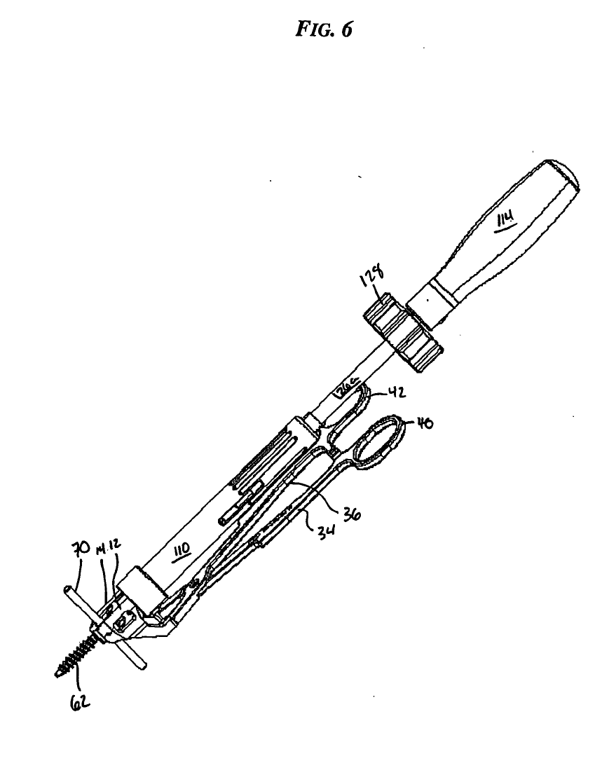

FIG. 6 illustrates the implant holder 10 and rod reduction device 100 in use with the spinal implant 50. The procedure first requires one or more spinal implants 50 to be secured within vertebral bone structures. Typically, where two spinal implants 50 are fastened into adjacent vertebrae, a spinal rod 70 is inserted into the rod-receiving portion 69 of each implant 50. Due to the alignment of the implants 50, however, it can be difficult to fully seat the rod 70 within each rod-receiving recess 109. Thus, the implant holder 10 is moved into the open position, using the actuating member 18, and the gripping arms 12, 14 are positioned around the head 60 of the implant 50 such that the spinal rod extends between the gripping arms 12, 14. The handle members 34, 36 of the actuating member 18 are then moved to the closed position to cause the gripping arms 12, 14, to engage the head 60 of the implant 50. The ratchet mechanism 38 of the implant holder 10 can be used to maintain the implant holder 10 in the closed position.

Once the implant holder 10 is firmly attached to the implant, the rod reduction device 100 can be attached to the proximal end 12a, 14a of the gripping arms 12, 14 by rotating the outer sleeve 110 to cause the luer lock elements 124, 116a, 116b on the outer sleeve 110 and the gripping arms 12, 14 to engage. In this configuration, the reduction cowl 106 is positioned between the gripping arms 12, 14 just above the spinal rod 70, and the rod reduction device 100 is axially aligned with the head 60 of the implant 50. The rod 70 can now be reduced into the rod-receiving portion 69 of the head 60 of the implant 50 by rotating the knob 128 on the pusher shaft 112 to thread the pusher shaft 112 in a distal direction with respect to the outer sleeve 110. As the pusher shaft 112 moves distally, the fastener-retaining member 104 with the fastener disposed therein may contact the rod 70 first. Further distal movement of the pusher shaft 112, however, will advance the reduction cowl 106 to a position in which the reduction cowl 106 is at least even with, if not distally beyond, the fastener-retaining member 104. When the rod 70 is fully reduced, the fastener on the distal end of the inserter tool 102 will enter the head 60 of the spinal implant 50. The handle member 114 on the inserter tool 102 can then be rotated to apply the fastener member to the head 60 of the implant 50. The “floating” or biased configuration of the reduction cowl 106 will allow the inserter tool 102 to move in a proximal-distal direction with respect to the reduction cowl 106. The tool 102 can thus be rotated, using handle 114, until the fastener is properly aligned with the head 60 of the implant 50. As a result, cross-threading of the fastener with the implant head 60 is avoided. Preferably, the inserter tool 102 is rotated in a counter-clockwise direction until the fastener drops into alignment with the fastener-receiving member, as will be indicated by a “click.” Once the fastener is in alignment with the fastener-receiving member, the inserter tool 102 can be rotated clockwise to fully thread the fastener into the head 60 of the implant 50, thereby locking the rod 70 to the implant 50.

One skilled in the art will appreciate further features and advantages of the invention based on the above-described embodiments. Accordingly, the invention is not to be limited by what has been particularly shown and described, except as indicated by the appended claims. All publications and references cited herein are expressly incorporated herein by reference in their entirety.

What is claimed is:

1. A spinal implant holder, comprising:
first and second gripping arms having a proximal portion with a mating element formed thereon for selectively mating to a medical device, and a distal portion adapted to at least partially circumferentially engage and axially align with a rod-receiving member of a spinal implant; and
an actuator mechanism coupled to the first and second gripping arms and effective to move the gripping arms between an open position, in which the gripping arms can be positioned around the rod-receiving member of a spinal implant, and a closed position, in which the gripping arms engage the rod-receiving member of the spinal implant.

2. The spinal implant holder of claim 1, wherein the first and second gripping arms are opposed to one another and each is substantially hemispherical and elongated between a proximal end and a distal end.

3. The spinal implant holder of claim 1, wherein the first and second gripping arms are effective to seat a rod reduction device used in spinal surgery, and to align the rod reduction device with the rod-receiving member of a spinal implant being engaged by the first and second gripping arms.

4. The spinal implant holder of claim 3, wherein each of the first and second gripping arms includes an inner, substantially hemispherical surface.

5. The spinal implant holder of claim 1, wherein each gripping arm includes an opening extending between the proximal and distal portions thereof to provide visual access to a spinal implant being engaged by the gripping arms.

6. The spinal implant holder of claim 1, wherein each gripping arm includes a grasping element formed on an inner surface thereof and effective to grasp a spinal implant.

7. The spinal implant holder of claim 6, wherein each grasping element comprises a pin member adapted to be disposed within a corresponding detent formed in the rod-receiving member of a spinal implant.

8. The spinal implant holder of claim 6, wherein each grasping element comprises a channel adapted to engage a corresponding lip formed around the rod-receiving member of a spinal implant.

9. The spinal implant holder of claim 1, wherein the mating element comprises a luer lock formed on at least one of an inner surface or an outer surface of the proximal portion of the first and second gripping arms.

10. The spinal implant holder of claim 1, further comprising a locking mechanism coupled to the actuator and effective to lock the first and second gripping arms in the closed position.

11. The spinal implant holder of claim 1, wherein the actuator comprises a first handle member mated to the first gripping arm, and a second handle member mated to the second gripping arm, the first and second handle members being pivotally coupled to one another and movable between a first, open position in which the gripping arms are in the open position, and a second, closed position in which the gripping arms are in the closed position.

12. The spinal implant holder of claim 11, further comprising a locking mechanism coupled to the first and second handle members and effective to lock the first and second handle members in the closed position, thereby locking the gripping arms in the closed position.

13. The spinal implant holder of claim 12, wherein the locking mechanism comprises a ratchet-type mechanism extending between the first and second handle members.

14. A rod reduction device, comprising:
an elongate inserter tool including a proximal end and a distal end having a fastener-retaining member formed thereon and adapted to retain a fastener therein, and a reduction cowl disposed around and biased to a proximal position with respect to the fastener-retaining member; and
an external housing disposed around at least a portion of the elongate inserter tool and including a first member adapted to couple to a rod-receiving member of a spinal implant, and a second member movable with respect to the first member and effective to apply a distally-directed force to the reduction cowl to advance a spinal rod disposed between the reduction cowl and the rod-receiving member of a spinal implant into the rod-receiving member of the implant;
wherein the reduction cowl is effective to enable the fastener-retaining member to move a predetermined distance in a proximal-distal direction with respect to the reduction cowl during reduction of a spinal rod.

15. The device of claim 14, wherein the first member of the external housing comprises an outer sleeve having a substantially cylindrical shape and including a proximal end, a distal end, and an inner lumen extending therethrough to receive the elongate inserter tool.

16. The device of claim 15, wherein the first member of the external housing includes opposed, flexible gripping arms that are adapted to mate to and grasp a rod-receiving portion of a spinal implant.

17. The device of claim 16, wherein the reduction cowl includes opposed finger-like members that are adapted to extend between legs of a U-shaped, rod-receiving head of a spinal implant.

18. The device of claim 15, wherein the first member of the external housing includes a mating element effective to mate to a spinal implant holder that is effective to couple to a rod-receiving portion of a spinal implant.

19. The device of claim 18, wherein the mating element comprises a luer lock formed on at least one of an inner surface or an outer surface of the first member of the external housing.

20. The device of claim 14, wherein the first and second members of the external housing are threadably mated to one another.

21. The device of claim 14, wherein the second member of the external housing comprises an elongate pusher shaft extending through the first member and having a distal end positioned adjacent to the reduction cowl.

22. The device of claim 21, wherein the elongate pusher shaft includes a proximal end having a handle formed thereon for effecting rotation movement of the pusher shaft.

23. An inserter tool disposable through a rod reduction device including a coupling member adapted to mate to a rod-receiving member of a spinal implant, and a pusher member axially adjustable with respect to the coupling member, the inserter tool comprising:
a shaft having a fastener-retaining member formed on a distal end thereof and a reduction cowl disposed around and biased in a proximal direction with respect to the fastener-retaining member, the reduction cowl being effective to move distally in response to a distally-directed force from the pusher member, greater than a biasing force, to advance a spinal rod toward a spinal implant mated to the coupling member, and to allow the fastener-retaining member to move in a proximal-distal direction during reduction of a spinal rod.

24. The inserter tool of claim 23, further comprising a spring disposed between the reduction cowl and the fastener-retaining member to bias the reduction cowl in a proximal direction.

25. The inserter tool of claim 23, wherein the fastener-retaining member includes an engagement feature formed on a distal end thereof for receiving and engaging a fastener.

26. The inserter tool of claim 23, wherein the fastener-retaining member comprises a cylindrical member defining prongs extending distally therefrom and effective to receive and engage a fastener.

27. The inserter tool of claim 23, wherein the reduction cowl comprises a hollow cylindrical member having an open distal end and a proximal end having a bore formed therein to slidably receive the shaft.

28. An implant holder and rod reduction system, comprising:
an implant holder having
first and second gripping members, each gripping member including a proximal portion having a mating element formed thereon, and a distal portion having a grasping element adapted to engage a rod-receiving member of a spinal implant, and
an actuating member effective to move the first and second gripping members between an open position, in which the members can be positioned around, but not in contact with, at least a portion of the rod-receiving member of a spinal implant, and a closed position, in which the members engage the rod-receiving member of the spinal implant; and
a rod reduction device having
a first member having a mating element formed thereon and adapted to removably mate to the mating element formed on each of the first and second gripping members, and
a second member rotatably coupled to and axially movable with respect to the first member such that the second member is effective to reduce a rod extending between the first and second gripping members and the spinal implant being engaged by the gripping members into the rod-receiving member of the spinal implant.

29. The system of claim 28, wherein the first and second gripping members are opposed to one another and each is substantially hemispherical and elongated between a proximal end and a distal end.

30. The system of claim 28, wherein the first and second gripping members are effective to axially align the rod reduction device with the rod-receiving member of a spinal implant being engaged by the first and second gripping arms.

31. The system of claim 30, wherein each of the first and second gripping members includes an inner, substantially hemispherical surface.

32. The system of claim 28, wherein the grasping element on each of the first and second gripping arms comprises a pin member adapted to be disposed within a corresponding detent formed in the rod-receiving member of a spinal implant.

33. The system of claim 32, wherein each grasping element comprises a channel adapted to engage a corresponding lip formed around the rod-receiving member of a spinal implant.

34. The system of claim 28, wherein the mating element on the first member and on each of the first and second gripping arms comprises a luer lock.

35. The system of claim 28, wherein further comprising a first handle member mated to the first gripping member, and a second handle member mated to the second gripping member, the first and second handle members being pivotally coupled to one another and movable between a first, open position in which the gripping members are in the open position, and a second, closed position in which the gripping members are in the closed position.

36. The system of claim 35, further comprising a locking mechanism coupled to the first and second handle members and effective to lock the first and second handle members in the closed position, thereby locking the gripping members in the closed position.

37. The system of claim 36, wherein the locking mechanism comprises a ratchet-type mechanism extending between the first and second handle members.

38. The system of claim 28, wherein the first member of the rod reduction device comprises an outer sleeve having a substantially cylindrical shape and including a proximal end, a distal end, and an inner lumen extending therethrough to receive the second member.

39. The system of claim 28, wherein the second member of the external housing comprises an elongate pusher shaft extending through the first member.

40. The system of claim 39, further comprising a fastener-receiving member formed on a distal end of the elongate pusher shaft and adapted to retain a fastener therein.

41. The system of claim 40, further comprising a reduction cowl disposed around the fastener-receiving member of the elongate pusher shaft, the reduction cowl being biased in a proximal direction with respect to the fastener-receiving member.

42. The system of claim 28, wherein the first and second members of the rod reduction device are threadably mated to one another.

43. The system of claim 39, wherein the elongate pusher shaft includes a proximal end having a handle formed thereon for effecting rotation movement of the pusher shaft.

44. A method for reducing a rod disposed within or adjacent to a rod-receiving head of a spinal implant, comprising the steps of:
providing a rod reduction device having
an inserter tool with a fastener-retaining member formed on a distal end thereof, and
an outer housing slidably disposed around the inserter tool and having a first member adapted to coupled to the head of a spinal implant, and a second member adapted to move distally with respect to the first member;
loading a fastener into the fastener-retaining member of the inserter tool;
coupling the first member of the outer housing to the head of a spinal implant disposed within a vertebral bone structure and having a rod disposed therein or adjacent thereto;
rotating the second member of the outer housing with respect to the first member of the outer housing to move the second member distally and thereby reduce a rod disposed within or adjacent to the rod-receiving head of the spinal implant; and
actuating the inserter tool to apply the fastener to the fastener-retaining member.
